(19) United States
(12) Patent Application Publication
(10) Pub. No.: US 2020/0069190 A1
(43) Pub. Date: Mar. 5, 2020

(54) PATCH-TYPE SENSOR MODULE

(71) Applicant: AMOLIFESCIENCE CO., LTD., Seoul (KR)

(72) Inventors: Kyung Hyun RYU, Seoul (KR); Beom Jin KIM, Bucheon-si, Gyeonggi-do (KR)

(73) Assignee: AMOLIFESCIENCE CO., LTD., Seoul (KR)

(21) Appl. No.: 16/468,461

(22) PCT Filed: Dec. 12, 2017
(86) PCT No.: PCT/KR2017/014510

§ 371 Date: Jun. 11, 2019

(30) Foreign Application Priority Data
Dec. 13, 2016 (KR) 10-2016-0169608
Feb. 9, 2017 (KR) 10-2017-0018178

(51) Int. Cl. A61B 5/01; H05K 1/18; H01Q 9/04; H01Q 1/27; H01Q 1/24; G01K 13/00; A61B 5/00

(52) U.S. Cl. CPC A61B 5/01; H05K 1/18; H01Q 9/0407; H01Q 1/273; H01Q 1/248; A61B 2560/0214; A61B 5/0008; A61B 5/6833; H05K 2201/10151; H05K 2201/09027; A61B 2562/0271; G01K 13/002

(57) ABSTRACT

A patch-type sensor module is provided. A patch-type sensor module according to one embodiment of the present invention comprises: a base substrate having flexibility and air permeability; an antenna pattern disposed on a first surface of the base substrate; a medicinal solution layer including a functional material and disposed on a second surface of the base substrate; a circuit board electrically connected to the antenna pattern, having at least one driving chip mounted thereon, and disposed on the first surface; and a temperature sensor mounted on the circuit board so as to sense a body temperature of a user.

### TECHNICAL FIELD

The present invention relates to a patch-type sensor module.

### BACKGROUND ART

Biological signals are signals representing physical conditions of humans and may be used to diagnose disease or health conditions.

These biological signals include electrical signals representing an electrocardiogram, an electroencephalogram, and an electromyogram, physical signals representing blood pressure, body temperature, and a pulse wave, and signals relating to compositions representing blood glucose contents, oxygen saturation, and body composition.

The biological signals are measured through sensors attached to skin. However, since not only oil and moisture, such as sweat and sebum, are present on human skin but also flection, such as a wrinkle, is formed on the human skin, there is a problem in that a sensor is not easily adhered to the human skin.

Further, when the sensor is attached to skin for a long period of time, since air permeability is not secured in an area in which the sensor is attached, there is a problem in that skin problems occur due to air blockage.

### DISCLOSURE

### Technical Problem

The present invention is directed to providing a patch-type sensor module capable of being easily attached to a human body and preventing skin problems.

Further, the present invention is directed to providing a patch-type sensor module capable of simultaneously implementing not only an inherent function of measuring a biological signal but also other additional functions.

Furthermore, the present invention is directed to providing a patch-type sensor module capable of being implemented in a thin shape by omitting a battery for driving a sensor.

### Technical Solution

One aspect of the present invention provides a patch-type sensor module including a base substrate having flexibility and air permeability, an antenna pattern disposed on a first surface of the base substrate, a medicinal solution layer including a functional material and disposed on a second surface of the base substrate, a circuit board electrically connected to the antenna pattern, having at least one driving chip mounted thereon, and disposed on the first surface, and a temperature sensor mounted on the circuit board so as to sense a body temperature of a user.

The base substrate may be formed of a nanofiber web of a three-dimensional network structure having micropores for blocking moisture and allowing air to pass therethrough. The nanofiber web may be formed by electrically spinning a spinning solution in which a synthetic polymer and a solvent are mixed. The medicinal solution layer may be formed of a nanofiber web accumulated by electrically spinning a spinning solution in which a functional material, a water-soluble polymer, and a solvent are mixed, and the functional material may include a dry storage material which is difficult to store in a liquid phase.

The antenna pattern may simultaneously perform a data transmission function of transmitting information obtained through the temperature sensor and a power reception function for supplying driving power required for the driving chip through an energy harvesting method.

The antenna pattern may be formed in a pattern on the first surface of the base substrate or on an upper portion of an insulating layer formed in a pattern on the first surface of the base substrate.

For example, in a case in which the antenna pattern is formed in a pattern on the first surface of the base substrate, the circuit board may be attached to the first surface of the base substrate via an anisotropic conductive film, and two terminals formed on both end portions of the antenna pattern may be electrically connected to the circuit board via the anisotropic conductive film.

In this case, the circuit board may include a first portion on which the at least one driving chip is mounted and a second portion extending from the first portion to cross the antenna pattern, and the two terminals of the antenna pattern may be electrically connected to the second portion.

Alternatively, the antenna pattern may include a bridge pattern formed from any one end portion of the both end portions to cross the antenna pattern, and any one of the two terminals is formed on an end portion of the bridge pattern. The bridge pattern may be insulated from the antenna pattern via an insulating layer disposed to surround the antenna pattern.

Alternatively, in a case in which the antenna pattern is formed in a pattern on an upper portion of an insulating layer which is formed and patterned on the first surface of the base substrate, the insulating layer may be formed in the same pattern as the antenna pattern.

The antenna pattern may be formed on the circuit board. In this case, the circuit board may be detachably attached to one surface of the base substrate via an adhesive member, and a shape holding member configured to maintain a shape of the base substrate may be attached to at least one surface of the base substrate along an edge of the base substrate. Consequently, the circuit board which is relatively expensive and electronic components mounted thereon can be reused.

An exposure hole may be formed to pass through the base substrate and the medicinal solution layer in a region corresponding to the temperature sensor, and the temperature sensor may be exposed to the outside through the exposure hole.

The circuit board may be prevented from being exposed to the outside through a protective member.

Another aspect of the present invention provides a patch-type sensor module including a base substrate formed of a nanofiber web of a three-dimensional network structure having micropores and having an antenna pattern formed on a first surface of the base substrate, a medicinal solution layer formed of a nanofiber web accumulated by electrically spinning a spinning solution in which a functional material, a water-soluble polymer, and a solvent are mixed and disposed on a second surface of the base substrate, a circuit board including a first portion on which at least one driving chip is mounted and a second portion extending from the first portion to cross the antenna pattern and to be electrically connected to the antenna pattern and attached to the first surface of the base substrate via an anisotropic conductive film, a temperature sensor mounted on the circuit board so as to sense a body temperature of a user and disposed in an exposure hole simultaneously passing through the base substrate and the medicinal solution layer, and a protective member configured to prevent the circuit board from being exposed to the outside.

Still another aspect of the present invention provides a patch-type sensor module including a base substrate formed of a nanofiber web of a three-dimensional network structure having micropores and having an antenna pattern formed on a first surface of the base substrate, a circuit board having at least one driving chip mounted thereon and attached to a first surface of the base substrate via an anisotropic conductive film so as to be electrically connected to the antenna pattern, a medicinal solution layer formed of a nanofiber web accumulated by electrically spinning a spinning solution in which a functional material, a water-soluble polymer, and a solvent are mixed and disposed on a second surface of the base substrate, a temperature sensor mounted on the circuit board so as to sense a body temperature of a user and disposed in an exposure hole formed to simultaneously pass through the base substrate and the medicinal solution layer, and a protective member configured to prevent the circuit board from being exposed to the outside.

### Advantageous Effects

In accordance with the present invention, a base substrate is formed of a nanofiber web having micropores such that flexibility and air permeability can be secured. Consequently, a patch-type sensor module according to the present invention can be easily attached to a human body due to flexibility in a state of being in close contact with the human body and, even when the patch-type sensor module is attached to a skin for a long period of time, air can be continuously supplied to an attachment portion of the skin such that side effects such as skin problems due to air blockage and the like can be prevented.

In addition, the patch-type sensor module according to the present invention can implement a skin improvement effect through a medicinal solution layer formed on one surface of the base substrate such that it is possible to obtain biometric data as well as a skin improvement effect through a functional material.

Further, since driving power is supplied to the patch-type sensor module according to the present invention using an energy harvesting method, it is unnecessary to use a battery which is conventionally used such that it is possible to implement the patch-type sensor module in a thin shape by omitting a space in which the battery is mounted.

### DESCRIPTION OF DRAWINGS

FIGS. 1A and 1B are schematic diagrams illustrating a top surface and a bottom surface of a patch-type sensor module according to a first embodiment of the present invention.

FIG. 2 is a diagram illustrating a state in which a circuit board is separated from a base substrate in FIG. 1.

FIG. 3 is a cross-sectional view taken along line A-A of FIG. 1.

FIGS. 4A and 4B are schematic diagrams illustrating a top surface and a bottom surface of a patch-type sensor module according to a second embodiment of the present invention.

FIG. 5 is a diagram illustrating a state in which a circuit board is separated from a base substrate in FIG. 5.

FIG. 6 is a cross-sectional view taken along line B-B of FIG. 5.

FIG. 7 is a schematic diagram illustrating a case in which an antenna pattern is formed on a base substrate in a patch-type sensor module according to the present invention.

FIGS. 8A and 8B are schematic diagrams illustrating a top surface and a bottom surface of a patch-type sensor module according to a third embodiment of the present invention.

FIG. 9 is an exploded view of FIG. 8.

FIG. 10 is a cross-sectional view taken along line B-B of FIG. 8.

FIG. 11 is a schematic diagram illustrating a base substrate applied to a patch-type sensor module according to the present invention.

### MODES OF THE INVENTION

Hereinafter, embodiments of the present invention will be fully described in detail which is suitable for easy implementation by those skilled in the art to which the present invention pertains with reference to the accompanying drawings. The present invention may be implemented in various different forms, and thus it is not limited to the embodiments which will be described herein. In the drawings, some portions not related to the description will be omitted in order to clearly describe the present invention, and the same or similar reference numerals are given to the same or similar components throughout this disclosure.

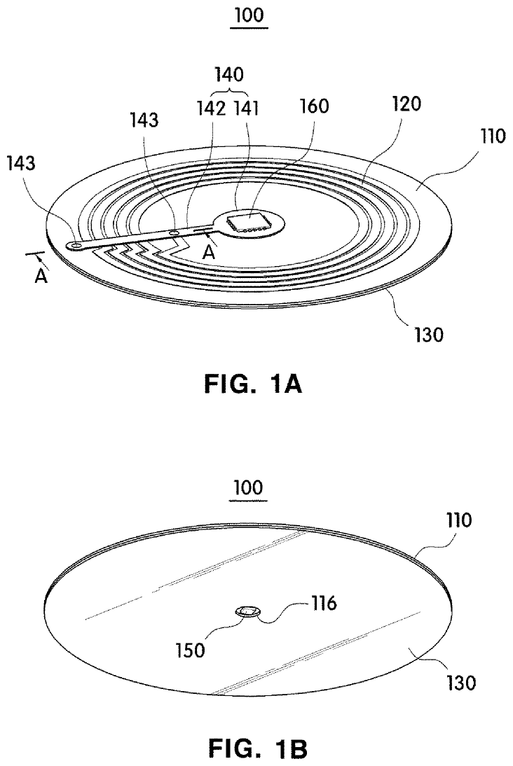

As shown in FIGS. 1A, 4A, and 8A, each of patch-type sensor modules 100, 200, and 300 according to one embodiment of the present invention include a base substrate 110, an antenna pattern 120, a medicinal solution layer 130, a circuit board 140 or 240, and a temperature sensor 150.

The base substrate 110 may support the medicinal solution layer 130 and the circuit board 140 or 240, which are each disposed on one of both surfaces of the base substrate 110. To this end, the base substrate 110 may be in the form of a plate including a first surface and a second surface which have a predetermined area. For example, the circuit board 140 or 240 may be disposed on the first surface, and the medicinal solution layer 130 may be disposed on the second surface. In the present invention, the first surface and the second surface may be opposite surfaces formed on the base substrate 110.

In this case, the base substrate 110 applied to the present invention may have flexibility, moisture blockability, and air permeability. To this end, the base substrate 110 may be formed of a nanofiber web having micropores 114.

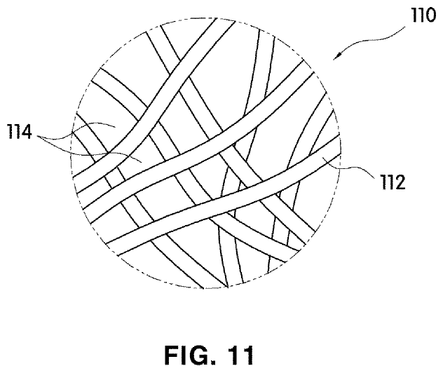

For example, as shown in FIGS. 7 and 11, the base substrate 110 may be a nanofiber web in which nanofibers 112 including a synthetic polymer are accumulated. That is, in order to allow air to freely pass through while blocking movement of moisture, the base substrate 110 may be formed of the nanofiber web having the micropores 114, and the nanofiber web may be formed in a three-dimensional network structure. In this case, an average pore diameter of the micropores 114 may be 10 μm or less.

Specifically, the base substrate 110 may be a single-layer nanofiber web accumulated so as to have the micropores 114 by electrically spinning a spinning solution in which a synthetic polymer and a solvent are mixed. Here, the solvent may be water or alcohol. Alternatively, in addition to the water or alcohol, the solvent may be an organic solvent.

In this case, the synthetic polymer may be a fiber-formable polymer capable of being electrically spun while not being dissolved by the solvent so as to implement a nanofiber web through electric spinning. Thus, even when the base substrate 110 is in contact with the solvent, the base substrate 110 may be not dissolved by the solvent and maintains a form of the nanofiber web. Consequently, the base substrate 110 may be attached to the skin for a long period of time. Further, even when a long period of time is elapsed after the base substrate 110 is attached to the skin, air may smoothly flow to the skin of the user through the micropores 114 such that skin problems, such as becoming macerated, occurring due to air blockage may be prevented.

In addition, in a case in which the medicinal solution layer 130 which is water-soluble is disposed on one surface of the base substrate 110 and is released in a liquid phase or a gel phase by coming into contact with a solvent applied on the skin of the user, the base substrate 110 may maintain the form of the nanofiber web while not being dissolved by the solvent. Consequently, the base substrate 110 may serve to support the released medicinal solution layer 130. Therefore, the patch-type sensor modules 100, 200, and 300 according to the present invention may prevent effective components contained in the medicinal solution layer 130 from passing through the base substrate 110 to leak to the outside while supplying air from the outside to a body part of the user through the micropores formed in the base substrate 110, thereby promoting penetration of the effective components into the skin of the user.

Further, the base substrate 110 may be formed such that the average pore diameter of the micropores has 10 μm or less. Consequently, the base substrate 110 may diffuse reflection of light passing through the micropores. Accordingly, even though a functional material such as an ultraviolet blocking component is not added, the base substrate 110 may have an effect of blocking ultraviolet rays due to the micropores.

In the present invention, the synthetic polymer is not particularly limited as long as the synthetic polymer is a resin which can be dissolved in a solvent for electric spinning, can form nanofibers by electric spinning, and is not dissolved by the solvent. As a non-limiting example, the synthetic polymer may include one or more selected from among polyvinylidene fluoride (PVDF), poly (vinylidene fluoride-co-hexafluoropropylene), a perfluoropolymer, polyvinyl chloride, polyvinylidene chloride, a copolymer thereof, polyethylene glycol derivatives including polyethylene glycol dialkyl ether and polyethylene glycol dialkyl ester, polyoxide including poly(oxymethylene-oligo-oxyethylene), polyethylene oxide, and polypropylene oxide, copolymers including polyvinyl acetate, poly(vinylpyrrolidone-vinyl acetate), polystyrene, and polystyrene acrylonitrile, polyacrylonitrile copolymers including polyacrylonitrile (PAN) and polyacrylonitrile methyl methacrylate, and a polymethyl methacrylate copolymer, or a mixture of two or more selected from thereamong.

The antenna pattern 120 may be formed in a predetermined pattern and may serve to transmit information obtained through the temperature sensor 150 to other external devices. In the present invention, the external device may be a portable electronic device such as a smart phone, a tablet personal computer (PC), or the like.

To this end, a pair of terminals 122a and 122b formed on both ends of the antenna pattern 120 may be electrically connected to the circuit board 140 or 240 and driven by a driving chip 160 mounted on the circuit board 140 or 240. Accordingly, the antenna pattern 120 may serve as a radiator for transmitting the information obtained through the temperature sensor 150 to the outside through a wireless communication technique.

In the present invention, all known wireless communication such as near field communication (NFC), Bluetooth communication, radio frequency identification (RFID) communication, infrared data association (IrDA) communication, ultra wideband (UWB) communication, ZigBee communication, long-range (LoRa) communication, RADAR communication, and low-power wireless communication may be used as the wireless communication technique.

The antenna pattern 120 may be formed in a pattern on one surface of the base substrate 110 or formed in a pattern on the circuit board 240.

In the present invention, each of the circuit boards 140 and 240 may be a double-sided board having circuit patterns formed on both sides thereof so as to allow the temperature sensor 150 and the driving chip 160 to be respectively mounted on opposite surfaces of the circuit board 140 or 240. Further, the temperature sensor 150 may be mounted on a second surface of the circuit board 140 or 240, which is an opposite surface to a first surface on which the driving chip 160 is mounted. In addition, each of the circuit boards 140 and 240 may be a flexible circuit board or a rigid circuit board.

For example, as shown in FIGS. 1A to 7, the antenna pattern 120 may be formed in a pattern on one surface of the base substrate 110. That is, the antenna pattern 120 may be formed in a predetermined pattern on one surface of the base substrate 110 by printing a conductive material thereon. As a non-limiting example, the conductive material may be an Ag paste or a Cu paste.

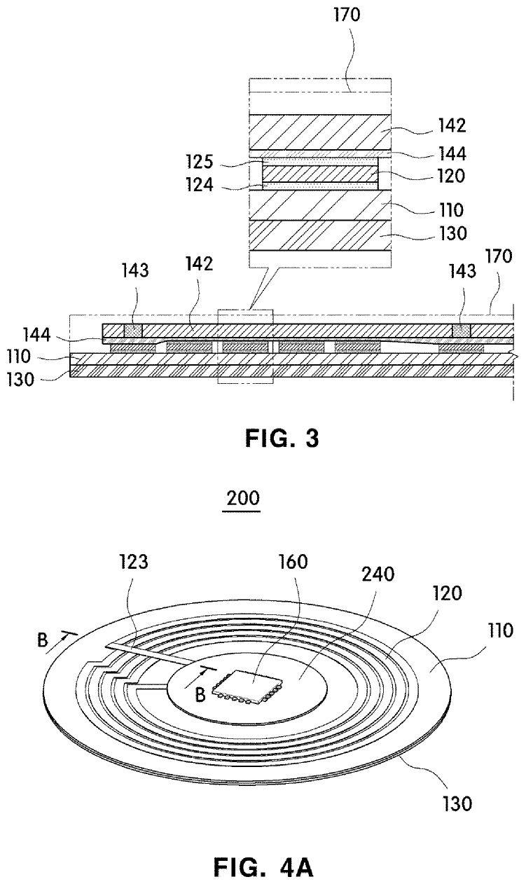

As shown in FIG. 3, the antenna pattern 120 may be formed in a pattern on one surface of an insulating layer 124, which is formed in a pattern on one surface of the base substrate 110, so as to prevent an electrical short circuit.

Here, as shown in FIG. 7, the insulating layer 124 may have a form of completely or partially filling the micropores 114 formed in the base substrate 110 or may have a form of being attached to one surface of the base substrate 110. In this case, the insulating layer 124 may be formed in the same pattern as the antenna pattern 120 and may have a width which is equal to or relatively wider than a width of the antenna pattern 120. Further, in the patch-type sensor module 100 according to the present embodiment, another insulating layer 125 for preventing an electrical short circuit may also be formed on an upper surface of the antenna pattern 120. In addition, the two terminals 122a and 122b formed on both end portions of the antenna pattern 120 may be electrically connected to the circuit board 140, and driving of the antenna pattern 120 may be controlled by the driving chip 160 mounted on the circuit board 140.

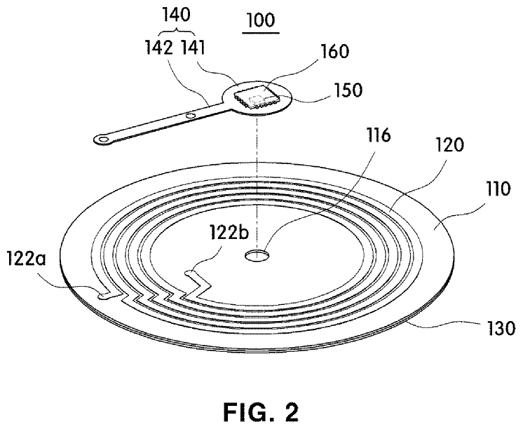

As a specific example, as shown in FIGS. 1A and 2, the circuit board 140 may include a first portion 141, on which the driving chip 160 and the temperature sensor 150 are mounted, and a second portion 142 extending from the first portion 141 and disposed to cross the antenna pattern 120. As shown in FIG. 3, the first portion 141 and the second portion 142 may be attached to one surface of the base substrate 110 via an adhesive member 144. As a non-limiting example, the adhesive member 144 may be a known anisotropic conductive film.

Accordingly, the second portion 142 may be electrically connected to parts of the terminals 122a and 122b of the antenna pattern 120 while maintaining insulation from the base substrate 110 and the antenna pattern 120.

That is, as shown in FIG. 3, via holes 143 may be formed in the circuit board 140 at positions corresponding to the two terminals 122a and 122b formed on the both end portions of the antenna pattern 120. The via holes 143 may be electrically connected to the two terminals 122a and 122b through an anisotropic conductive film that is the adhesive member 144. Further, upper sides of the via holes 143 may be electrically connected to the driving chip 160 through a circuit pattern formed on an upper surface of the circuit board 140.

In the present embodiment, the base substrate 110 may serve not only to support the medicinal solution layer 130 but also as a circuit board on which the antenna pattern 120 is formed. Thus, since the base substrate 110 may serve as the circuit board on which the antenna pattern 120 is formed by being formed of a nanofiber web in which nanofibers are accumulated, the base substrate 110 has excellent flexibility as compared with a conventional polyimide film generally used in a flexible circuit board and has an excellent restoring characteristic which is able to return to its original flat state even when folded or crumpled.

Further, since air permeability and moisture blockability are secured through the micropores 114 in the remaining area of the base substrate 110 except for an area in which the antenna pattern 120 is formed, even when the antenna pattern 120 is formed on one surface of the base substrate 110, the air permeability may be secured sufficiently.

However, the method of forming the antenna pattern 120 is not limited to the above description, and the antenna pattern 120 may be directly formed on one surface of the base substrate 110 by completely or partially filling the micropores 114 formed in the base substrate 110 with the conductive material.

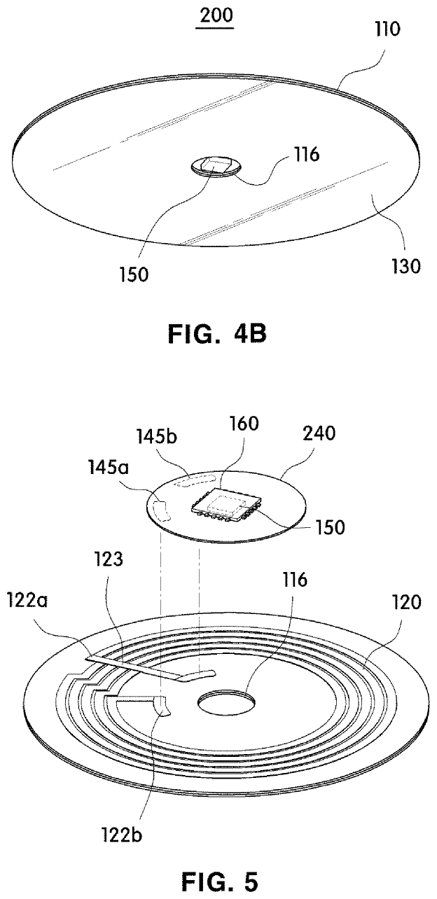

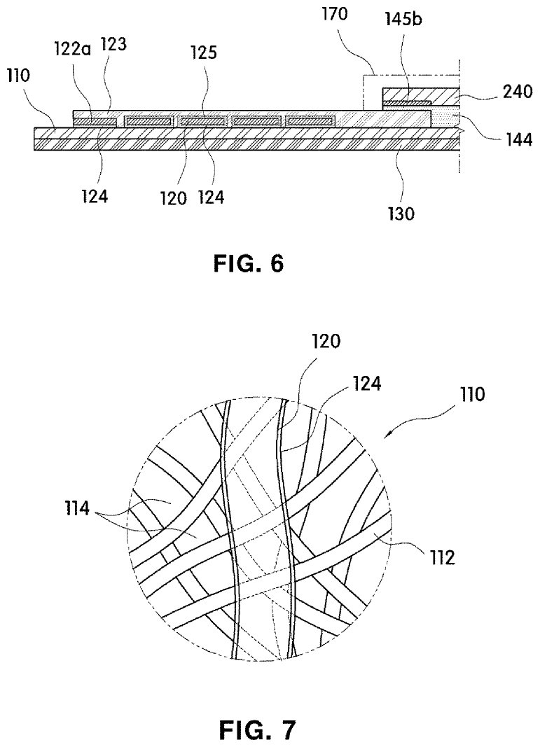

Alternatively, as shown in FIGS. 4A to 6, the antenna pattern 120 may be formed in a pattern on one surface of the base substrate 110. In this case, as shown in FIG. 6, the antenna pattern 120 may be formed in a pattern on one surface of the insulating layer 124 so as to prevent an electrical short circuit.

Here, as shown in FIG. 7, the insulating layer 124 may have a form of completely or partially filling the micropores 114 formed in the base substrate 110 or may have a form of being attached to one surface of the base substrate 110. Further, the insulating layer 124 may be formed in the same pattern as the antenna pattern 120 and may have a width which is equal to or relatively wider than a width of the antenna pattern 120.

In addition, the circuit board 240 may be attached to one surface of the base substrate 110 via the adhesive member 144. As a non-limiting example, the adhesive member 144 may be an anisotropic conductive film.

In this case, as shown in FIG. 6, the patch-type sensor module 200 according to the present embodiment may include another insulating layer 125 surrounding the top and side surfaces of the antenna pattern 120 so as to prevent an electrical short circuit of the antenna pattern 120. Accordingly, the antenna pattern 120 may be completely surrounded by the insulating layer 124 and 125, and even when still another circuit pattern 123 is formed on an upper side of the antenna pattern 120, still another circuit pattern 123 may maintain an insulating state from the antenna pattern 120.

Specifically, still another circuit pattern 123 may be formed to extend inward from the terminal 122a, which is formed on a relatively outer side of the pair of terminals 122a and 122b formed on the both end portions of the antenna pattern 120, to cross the antenna pattern 120. In this case, still another circuit pattern 123 may maintain insulation from the antenna pattern 120 through another insulating layer 125. Accordingly, unlike the above described first embodiment, still another circuit pattern 123 may serve as a bridge in the present embodiment.

Consequently, the terminal 122a formed on the outer side of the pair of terminals 122a and 122b, which are formed on the both end portions of the antenna pattern 120, may be moved to an inner hollow portion of the antenna pattern 120 through still another circuit pattern 123.

Thus, the pair of terminals 122a and 122b formed on the both end portions of the antenna pattern 120 may be directly electrically connected to the circuit board 240 disposed on the inner hollow portion of the antenna pattern 120, and driving of the antenna pattern 120 may be controlled by the driving chip 160 mounted on the circuit board 240. Here, as shown in FIG. 5, a pair of terminals 145a and 145b may be formed on a bottom surface of the circuit board 240 in regions corresponding to the terminal 122b and the terminal 124a formed on an end portion of still another circuit pattern 123. Accordingly, the terminal 122b and the terminal 124a formed on the end portion of still another circuit pattern 123 may be respectively electrically connected to the pair of terminals 145a and 145b formed on the circuit board 240 via an anisotropic conductive film which is the adhesive member 144.

Thus, in the present embodiment, even when the circuit board 240 is disposed in only the inner hollow portion of the antenna pattern 120, the circuit board 240 may be electrically connected to the terminals 122a and 122b of the antenna pattern 120. Consequently, a size of the circuit board 240 may be reduced such that a material cost may be reduced. Further, an area occupied by the circuit board 240 is reduced and thus an area of the base substrate 110 covered by the circuit board 240 may be reduced such that more excellent air permeability may be secured. In addition, the antenna pattern 120 is directly connected to the circuit board 240 through still another circuit pattern 123 serving as a bridge such that electrical reliability may be enhanced. Further, even when the circuit board 240 is made of a rigid material, flexibility may be secured in the remaining area of the base substrate 110 except for an area thereof corresponding to the circuit board 240.

In the present embodiment, the base substrate 110 may serve not only to support the medicinal solution layer 130 but also as a circuit board on which the antenna pattern 120 is formed. Thus, since the base substrate 110 may serve as the circuit board on which the antenna pattern 120 is formed by being formed of a nanofiber web in which nanofibers are accumulated, the base substrate 110 has excellent flexibility as compared with a conventional polyimide film generally used in a flexible circuit board and has an excellent restoring characteristic which is able to return to its original flat state even when folded or crumpled.

Further, since air permeability and moisture blockability are secured through the micropores 114 in the remaining area of the base substrate 110 except for an area in which the antenna pattern 120 is formed, even when the antenna pattern 120 is formed on one surface of the base substrate 110, the air permeability may be secured sufficiently.

However, the method of forming the antenna pattern 120 is not limited to the above description, and the antenna pattern 120 may be directly formed on one surface of the base substrate 110 by completely or partially filling the micropores 114 formed in the base substrate 110 with the conductive material.

In addition, the method of electrically connecting the terminal 122b and the terminal 124a formed on the end portion of still another circuit pattern 123 to the pair of terminals 145a and 145b is not limited to the above description, and the terminal 122b and the terminal 124a formed on the end portion of still another circuit pattern 123 may be electrically connected to the pair of terminals 145a and 145b through a direct contact method.

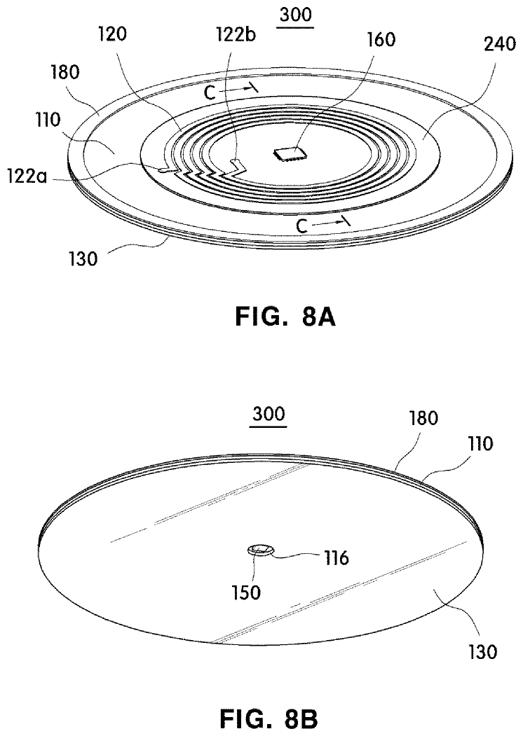

Alternatively, as shown in FIGS. 8A to 10, in the patch-type sensor module 300 according to one embodiment of the present invention, the antenna pattern 120 may be formed on the circuit board 240 on which the driving chip 160 and the temperature sensor 150 are mounted. In the present embodiment, the antenna pattern 120 may be formed in a predetermined pattern on one surface of the circuit board 240 and may be electrically connected to the driving chip 160.

Here, the circuit board 240 may be a double-sided board so as to allow the temperature sensor 150 and the driving chip 160 to be mounted on opposite surfaces of the circuit board 240. One of the two terminals 122a and 122b formed on both ends of the antenna pattern 120 may be connected to the driving chip 160 via a via hole and a lead portion. In addition, the circuit board 240 may be a flexible circuit board or a rigid circuit board.

In this case, one surface of the circuit board 240 may be detachably coupled to one surface of the base substrate 110 through an adhesive member 244. Further, the temperature sensor 150 may be mounted on one of both surfaces of the circuit board 240, which is an opposite surface to a surface on which the driving chip 160 is mounted. Here, the adhesive member 244 may be non-base material type member of a liquid phase or a gel phase or a base material type member having both surfaces coated with an adhesive material. Further, the adhesive member 244 may contain a non-conductive component for electrical insulation between the base substrate 110 and the circuit board 240.

That is, the patch-type sensor module 300 according to the present embodiment may be configured such that all of the antenna pattern 120, the driving chip 160, and the temperature sensor 150 are provided on a single circuit board 240, and the circuit board 240 may be attached to one surface of the base substrate 110.

Accordingly, when the base substrate 110 needs to be replaced, the circuit board 240 may be separated from the base substrate 110, and the separated circuit board 240 may be attached to another unused surface of the base substrate 110 through the adhesive member 244 again.

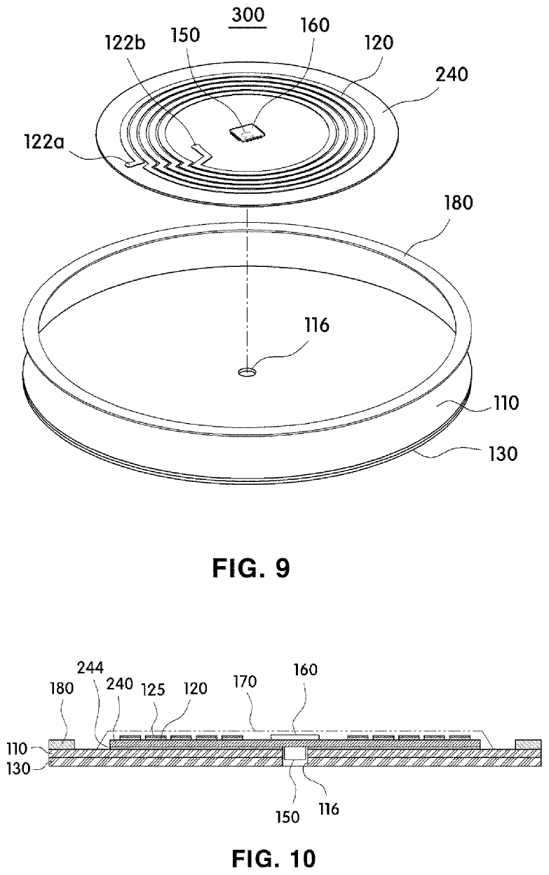

Consequently, the remaining portions except for the base substrate 110 and the medicinal solution layer 130 may be reused. In this case, as shown in FIGS. 8A and 9, at least one shape maintaining member 180 for maintaining a shape of the base substrate 110 may be attached to at least one surface of the base substrate 110 along an edge of the base substrate 110. Thus, ease of a separation operation for separating the circuit board 240 from the base substrate 110 may be enhanced.

Here, as a non-limiting example, the shape maintaining member 180 may be a fluoride resin-based film member such as polyethylene terephthalate (PET), but the present invention is not limited thereto, and it is noted that the shape maintaining member 180 may be formed of a metal material or a plastic material having rigidity.

Meanwhile, the antenna pattern 120 applied to the present invention may simultaneously perform a data transmission function of transmitting information obtained through the temperature sensor 150 and a power reception function for supplying driving power to the driving chip 160.

That is, the antenna pattern 120 may receive power from an external device using an energy harvesting method and supply power received from the external device to the driving chip 160.

For example, while serving as an NFC antenna for data transmission with other external devices such as a portable device, the antenna pattern 120 may receive wireless power for driving the driving chip 160 from an external device. Accordingly, since the patch-type sensor modules 100, 200, and 300 according to the present invention do not require a separate power source such as a battery which is typically embedded to drive the driving chip 160, a weight corresponding to the battery may be reduced. Further, since the battery which is a power supply source may be omitted from the patch-type sensor modules 100, 200, and 300 according to the present invention, a dimension and a thickness corresponding to a size of the battery may be reduced such that the patch-type sensor modules 100, 200, and 300 may be implemented in an ultra-thin shape.

The medicinal solution layer 130 is formed on one surface of the base substrate 110. The medicinal solution layer 130 may be in direct contact with the skin of the user to provide an advantageous effective component to the skin thereof. To this end, the medicinal solution layer 130 may be a nanofiber web formed to have micropores by electrically spinning a spinning solution in which a water-soluble polymer, a functional material, and a solvent are mixed at an appropriate ratio.

That is, the medicinal solution layer 130 may be implemented in the form of a nanofiber web through a spinning solution in which a water-soluble polymer material and a functional material are mixed. Accordingly, when the medicinal solution layer 130 is attached to the skin on which a solvent is applied and then is in contact with the solvent, the medicinal solution layer 130 may be changed into a released state. Consequently, the functional material contained in the medicinal solution layer 130 may be absorbed into the skin, and the water-soluble polymer material may be absorbed into the base substrate 110.

Here, the water-soluble polymer material is not particularly limited as long as it is a polymer material which is dissolved in water or alcohol and can form nanofibers through electric spinning As a non-limiting example, the water soluble polymer material may be a mixture including one or more selected from among polyvinyl alcohol (PVA), polyvinyl pyrrolidone (PVP), polyethylene oxide (PEO), carboxyl methyl cellulose (CMC), starch, polyacrylic acid (PAA), and hyaluronic acid.

Further, the functional material may be a dry storage material which is difficult to store in a liquid phase. In addition, when the water-soluble polymer is dissolved, the dry storage material may be released in a state of a liquid phase or a phase such that the dry storage material may be smoothly absorbed into the skin of the user. For example, the dry storage material may be a vitamin, an enzyme, a protein, a peptide-vitamin C derivative, or the like. Usually, the above-described dry storage material has a property of being decomposed only in a liquid phase. However, it is difficult to store the dry storage material in a liquid state for a long period of time.

In the present invention, the dry storage material which is difficult to store in a liquid phase is included in the spinning solution together with the water-soluble polymer material and the solvent, the spinning solution containing the dry storage material is formed into nanofibers through electric spinning, and the medicinal solution layer 130 is configured in the form of a nanofiber web such that the dry storage material may be bound in a dry state in the nanofibers constituting the medicinal solution layer 130.

Thus, the dry storage material which is difficult to store in a liquid phase may be stored for a long period of time, and, when the water-soluble polymer is dissolved by the solvent, the functional material in a dry state may be released together with the water-soluble polymer. Consequently, the functional material may be transferred to the skin and smoothly penetrate into the skin.

That is, when the patch-type sensor module 100, 200, or 300 according to the present invention is attached to the skin, the water-soluble polymer constituting the medicinal solution layer 130 may be dissolved by the solvent applied on the skin, and the functional material bound by the water-soluble polymer may be released. Accordingly, the released functional material may be absorbed into the skin, and the water-soluble polymer dissolved by the solvent may be absorbed into the base substrate 110.

In the present invention, the functional material is a material for skin care and wound care and may be a mixture including any one among ingredients that help skin whitening (arbutin, niacinamide, and ascoglucoside), ingredients that help to improve skin wrinkles (retinol and adenosine), an ingredient that helps to block ultraviolet rays (titanium dioxide), ingredients that aid in moisturizing and skin elasticity (a snail mucilage filtrate, acetyl hexapeptide, red ginseng collagen, aqua ceramide, regenerating peptide, and a galactomyces fermentation liquid), growth factors such as an epithelial growth factor (EGF) and a fibroblast growth factor (FGF), a protein for healing, and antimicrobial substances such as silver nano materials and chitosan. Alternatively, the functional material may be a mixture including one or more selected from among water-soluble collagen, vegetable platinum, tocopherol, xylitol, and a vegetable extract.

In this case, a predetermined ratio of oil may be contained in the spinning solution for forming the medicinal solution layer 130 so as to adequately control a time for which the medicinal solution layer 130 is dissolved when in contact with the solvent. Consequently, an overall drying time of the base substrate 110 attached to the skin of the user may be controlled so that the patch-type sensor modules 100, 200, and 300 according to the present invention may have a proper drying time suitable for various purposes such as sleeping, a mask pack, protection, and the like.

Accordingly, the patch-type sensor modules 100, 200, and 300 according to the present invention may supply the advantageous effective ingredient to the skin through the medicinal solution layer 130 while collecting information on a body temperature of the user through the temperature sensor 150 such that it is possible to simultaneously achieve information acquisition and an effect of skin improvement.

The temperature sensor 150 may be mounted on the circuit board 140 or 240 disposed on one surface of the base substrate 110 to sense the body temperature of the user.

As described above, the temperature sensor 150 may be mounted on one of the both surfaces of the circuit board 140 or 240 opposite to the other surface on which the driving chip 160 is mounted. Accordingly, when the patch-type sensor module 100, 200, or 300 according to the present invention is attached to the skin of the user, the temperature sensor 150 may be exposed to the user's body.

To this end, an exposure hole 116 may be formed to pass through the base substrate 110 and the medicinal solution layer 130 at a region corresponding to the temperature sensor 150. Accordingly, when the circuit board 140 or 240 is attached on one surface of the base substrate 110 in a state in which the temperature sensor 150 and the driving chip 160 are mounted on the both surfaces of the circuit board 140 or 240, the temperature sensor 150 may be inserted into the exposure hole 116. When the patch-type sensor module 100, 200, or 300 according to the present invention is attached to the user's body, the temperature sensor 150 may face the skin of the user to measure a body temperature of the user.

Further, as described above, valid information generated based on the information sensed from the temperature sensor 150 may be transmitted to the outside through the antenna pattern 120.

Meanwhile, as shown in FIGS. 3, 6, and 10, the patch-type sensor modules 100, 200, or 300 according to the present invention may include a protective member 170 to protect the circuit board 140 or 240 and/or the driving chip 160 from being exposed to the outside. For example, the protective member 170 may be formed of a fluoropolymer resin such as PET, polypropylene (PP), polyethylene (PE), or the like or may be in the form of a sheet such as release paper or in the form of a molding covered with a resin material made as an insulator.

Here, the protective member 170 may be in the form of partially covering the circuit board 140 or 240 and/or the driving chip 160. In particular, the protective member 170 may be in the form of covering a region corresponding to the driving chip 160. However, the covering region of the protective member 170 is not limited thereto, and the protective member 170 may be provided in the form of having an area that is substantially equal to an area of the base substrate 110 to cover all of the circuit board 140 or 240 and the antenna pattern 120.

Further, in the patch-type sensor module 100, 200, or 300 according to the present invention, a known shielding sheet (not shown) may be disposed in a region corresponding to the antenna pattern 120 so as to prevent influence of an eddy current by shielding a magnetic field generated from the antenna pattern 120, and a heat insulating sheet may be included so as to prevent heat generated in the driving chip 160 from being transmitted to a human body.

Here, all known magnetic materials used for a shielding sheet, such as ferrite, an amorphous material, polymer, and the like, may be used in the shielding sheet, and the heat insulating sheet may be in the form of a metal or graphite sheet or in a form in which a nano web and a metal are stacked.

Meanwhile, in the above-described embodiments, although the functional material has been described as being contained in only the medicinal solution layer 130, the present invention is not limited thereto, and the functional material may be included in the base substrate 110. That is, in addition to a synthetic polymer material and a solvent for maintaining the shape of the nanofiber web in the base substrate 110, the spinning solution may further include the functional material.

Further, the patch-type sensor modules 100, 200, and 300 according to the present invention may be implemented in a form in which the medicinal solution layer 130 is omitted such that the patch-type sensor modules 100, 200, and 300 may be used for a sensor for simply sensing the body temperature of the user.

In addition, it is noted that, in the present invention, the spinning method of forming the base substrate 110 and the medicinal solution layer 130 may employ any one of general electric spinning, air spinning, electrospraying, electroblown spinning, centrifugal electrospinning, and flash spinning

The above-described patch-type sensor modules 100, 200, and 300 according to the present invention may each be implemented as a healthcare product or a medical product. Further, it is noted that the patch-type sensor modules 100, 200, and 300 according to the present invention may be applied to not only clothing products such as vests, shoes, clothes, and the like but also wearable devices such as a smart watch and a smart glass and may further be applied to a mask pack and the like.

In addition, although the temperature sensor is exemplified as a kind of sensor in the present invention, the present invention is not limited thereto, and it is noted that the temperature sensor may be replaced with a known biosensor to measure biometric data such as body fat, skeletal muscle mass, heart rate, electrocardiogram, stress response, electroencephalogram, blood flow rate, electromyogram, and the like.

Although the exemplary embodiments of the present invention have been described, the spirit of the present invention is not limited to the exemplary embodiments disclosed herein, and it should be understood that numerous other embodiments can be devised by those skilled in the art that will fall within the same spirit and scope of this disclosure through addition, modification, deletion, supplement, and the like of a component, and also these other embodiments will fall within the spirit and scope of the present invention.

1. A patch-type sensor module comprising:
a base substrate having flexibility and air permeability;
an antenna pattern disposed on a first surface of the base substrate;
a medicinal solution layer including a functional material and disposed on a second surface of the base substrate;
a circuit board electrically connected to the antenna pattern, having at least one driving chip mounted thereon, and disposed on the first surface; and
a temperature sensor mounted on the circuit board so as to sense a body temperature of a user.

2. The patch-type sensor module of claim 1, wherein the base substrate is formed of a nanofiber web of a three-dimensional network structure having micropores for blocking moisture and allowing air to pass therethrough.

3. The patch-type sensor module of claim 2, wherein the nanofiber web is formed by electrically spinning a spinning solution in which a synthetic polymer and a solvent are mixed.

4. The patch-type sensor module of claim 1, wherein the medicinal solution layer is formed of a nanofiber web accumulated by electrically spinning a spinning solution in which a functional material, a water-soluble polymer, and a solvent are mixed.

5. The patch-type sensor module of claim 4, wherein the functional material includes a dry storage material which is difficult to store in a liquid phase.

6. The patch-type sensor module of claim 1, wherein the antenna pattern simultaneously performs a data transmission function of transmitting information obtained through the temperature sensor and a power reception function for supplying driving power required for the driving chip through an energy harvesting method.

7. The patch-type sensor module of claim 1, wherein:
the antenna pattern is formed in a pattern on the first surface of the base substrate;
the circuit board is attached to the first surface of the base substrate via an anisotropic conductive film; and
two terminals formed on both end portions of the antenna pattern are electrically connected to the circuit board via the anisotropic conductive film.

8. The patch-type sensor module of claim 7, wherein:
the circuit board includes a first portion, on which the at least one driving chip is mounted, and a second portion extending from the first portion to cross the antenna pattern; and
the terminals of the antenna pattern are electrically connected to the second portion.

9. The patch-type sensor module of claim 7, wherein:
the antenna pattern includes a bridge pattern formed from one end portion of the both end portions to cross the antenna pattern; and
one of the two terminals is formed on an end portion of the bridge pattern.

10. The patch-type sensor module of claim 9, wherein the bridge pattern is insulated from the antenna pattern via an insulating layer disposed to surround the antenna pattern.

11. The patch-type sensor module of claim 7, wherein the antenna pattern is formed in a pattern on an upper surface of an insulating layer which is formed in a pattern on the first surface of the base substrate.

12. The patch-type sensor module of claim 11, wherein the insulating layer is formed in the same pattern as the antenna pattern.

13. The patch-type sensor module of claim 1, wherein the antenna pattern is formed on the circuit board.

14. The patch-type sensor module of claim 13, wherein the circuit board is detachably attached to one surface of the base substrate via an adhesive member.

15. The patch-type sensor module of claim 13, wherein a shape holding member configured to maintain a shape of the base substrate is attached to at least one surface of the base substrate along an edge of the base substrate.

16. The patch-type sensor module of claim 1, wherein:
an exposure hole is formed to pass through the base substrate and the medicinal solution layer in a region corresponding to the temperature sensor; and
the temperature sensor is exposed to the outside through the exposure hole.

17. The patch-type sensor module of claim 1, wherein the circuit board is prevented from being exposed to the outside through a protective member.

18. A patch-type sensor module comprising:
a base substrate formed of a nanofiber web of a three-dimensional network structure having micropores and having an antenna pattern formed on a first surface of the base substrate;
a medicinal solution layer formed of a nanofiber web accumulated by electrically spinning a spinning solution in which a functional material, a water-soluble polymer, and a solvent are mixed and disposed on a second surface of the base substrate;
a circuit board including a first portion on which at least one driving chip is mounted and a second portion extending from the first portion to cross the antenna pattern and to be electrically connected to the antenna pattern and attached to the first surface of the base substrate via an anisotropic conductive film;
a temperature sensor mounted on the circuit board so as to sense a body temperature of a user, and disposed in an exposure hole simultaneously passing through the base substrate and the medicinal solution layer; and
a protective member configured to prevent the circuit board from being exposed to the outside.

19. A patch-type sensor module comprising:
a base substrate formed of a nanofiber web of a three-dimensional network structure having micropores and having an antenna pattern formed on a first surface of the base substrate;
a circuit board having at least one driving chip mounted thereon and attached to a first surface of the base substrate via an anisotropic conductive film so as to be electrically connected to the antenna pattern;
a medicinal solution layer formed of a nanofiber web accumulated by electrically spinning a spinning solution in which a functional material, a water-soluble polymer, and a solvent are mixed and disposed on a second surface of the base substrate;
a temperature sensor mounted on the circuit board so as to sense a body temperature of a user and disposed in an exposure hole formed to simultaneously pass through the base substrate and the medicinal solution layer; and
a protective member configured to prevent the circuit board from being exposed to the outside.
